(19) United States
(12) Patent Application Publication
(10) Pub. No.: US 2022/0378628 A1
(43) Pub. Date: Dec. 1, 2022

(54) MOISTURE, GAS AND FLUID-ENABLED SENSORS

(71) Applicant: AquaSensing Incorporated, Waterloo (CA)

(72) Inventors: Jiayun FENG, Waterloo (CA); Norman ZHOU, Waterloo (CA); George SHAKER, Waterloo (CA); Ming XIAO, Waterloo (CA); Walter DULEY, Waterloo (CA); Levi Nathan JOHNSTON, Sudbury (CA)

(21) Appl. No.: 17/775,943

(22) PCT Filed: Nov. 12, 2020
(86) PCT No.: PCT/CA2020/051527

§ 371 Date: May 11, 2022

Related U.S. Application Data
(60) Provisional application No. 62/934,175, filed on Nov. 12, 2019.
(60) Provisional application No. 62/934,182, filed on Nov. 12, 2019.
(60) Provisional application No. 62/934,190, filed on Nov. 12, 2019.

(51) Int. Cl. A61F 13/42; G01N 27/416; G01N 27/07

(52) U.S. Cl. CPC A61F 13/42; G01N 27/416; G01N 27/07; A61F 2013/424; B82Y 15/00

(57) ABSTRACT

A moisture, gas, fluid enabled sensor that includes an electronics component and a sensing component. The sensing component includes active electrode layer, a middle layer and a less active layer. When exposed to moisture, gas or fluid, the sensing component generates electricity which is then used to power the electronics component.

### CROSS-REFERENCE TO OTHER APPLICATIONS

The current disclosure claims priority from US Provisional Applications Nos. 62/934,190 filed Nov. 12, 2019; 62/934,175 filed Nov. 12, 2019 and 62/934,182 filed Nov. 12, 2019, the contents of which are hereby incorporated by reference.

### FIELD

The present disclosure is generally directed at sensors, and more specifically, at a self-powered sensor for detecting moisture, gas and fluids (such as, but not limited to, humidity, water, urine and blood) and method of manufacturing same.

### BACKGROUND

Sensing and detection of elements have a large range of applications in health diagnostics, industry process monitoring and environment protection. In fields requiring a controlled level of moisture or humidity, including electronic manufacturing, optical measurement and processing, nuclear applications, biomedical applications and vapor leakage detection, the sensor can send signals indicating the existence of moisture and vapor. In the fields of humidity level measurement, such as the detection of a breath pulse, this sensor provides a sensitive response to the generated moisture. In one simple example of health care, the breath frequencies of patients during sleeping are different according to the condition of their heart and throat, which can assist in the monitoring and diagnosis of potential diseases.

At present existing commercial humidity or moisture sensors are mostly powered by batteries, so their minimum volumes are limited, and the quantity of charges are also restricted by their volumes.

Therefore, there is provided a novel self-powered moisture, gas and fluid-enabling sensor and method of manufacturing same.

### SUMMARY

The disclosure is directed at a moisture, gas or fluid-enabled sensor. In one embodiment, the sensor may be seen as self-powering. The sensor includes an electronics component and a sensing component whereby the sensing component generates electricity or power, such as when exposed to moisture, gas or fluid. This generated electricity is then used by the electronics components to perform certain applications or functions.

The generation of power is based on an electrophysical and/or electrochemical reaction between an active metal electrode layer and humidity, fluid or moisture absorbed by a middle layer that is contact with the active metal electrode layer. In one embodiment, the middle layer is made from porous hydrophilic nano- or micro-scale materials. One advantage of the disclosure is that there is no need for external electrolytes to be added to the sensing component as the adsorption of moisture/fluid by the middle layer initiates the generation of electricity by the sensing component.

In one aspect of the disclosure, there is provided a self-powered sensing device including an electronics component; a sensing component, the sensing component including: an active material electrode layer; a less active electrode layer; a middle layer between the active material electrode layer and the less active layer, the middle layer incorporating at least one material with nano- and/or micro-scale structures; wherein electricity is generated by the sensing component to power the electronics component when moisture comes into contact with the middle layer.

In another aspect, the middle layer includes pressed graphite-based powder or graphite. In a further aspect, the pressed graphite powder is pressed into a disc-shaped middle layer. In yet another aspect, the middle layer is porous and hydrophilic. In an aspect, the active material electrode layer and the less active electrode layer are in direct electrical contact with the middle layer. In another aspect, absorption of moisture, gas or fluid by the middle layer generates a voltage difference between the active material electrode layer and the less active electrode layer.

In a further aspect, the middle layer includes carbon nanofibers (CNF), carbon nanoparticles (CNP), graphene flakes, graphite or TiO₂ nanowires. In another aspect, the middle layer is treated via a hydrophilic treatment. In yet a further aspect, the hydrophilic treatment includes an oxygen plasma treatment or acid oxidation. In another aspect, a material of the less active electrode layer is less chemically or physically reactive with respect to moisture compared to a material of the active material electrode layer. In yet another aspect, the active material electrode layer, the less active electrode layer and the middle layer comprise a single layer of a material or a multi-layer of the material. In another aspect, the active material electrode layer, the less active electrode layer and the middle layer include a single or multi-layer of a mixture of materials.

In another aspect, the electronics component includes at least one of a low-energy wireless device, a low-energy wireless communication device, a Bluetooth™ low energy (BLE) device and an application specific sensor. In a further aspect, the application specific sensor includes a humidity sensor, a lactate sensor, a mineral sensor, a temperature sensor, a glucose level sensor, a urine analysis component or a blood analysis component. In yet another aspect, the low-energy wireless device is powered by absorption of moisture by the middle layer generating a voltage difference between the active material electrode layer and the less active electrode layer. In yet a further aspect, the electronics component includes a radio component.

In another aspect, the active material electrode layer includes magnesium (Mg), Aluminium (Al), Iron (Fe), alloys of Mg, Al or Fe or other materials that facilitate a reaction between the active material electrode layer and moisture. In another aspect, the passive electrode layer includes copper or conductive materials which are less reactive with moisture than the active material electrode layer.

In another aspect of the disclosure, there is provided a system for moisture detection including at least one self-powered sensing devices, the at least one self-powered sensing devices including an electronics component; and a sensing component, the sensing component including an active material electrode layer; a less active electrode layer; a middle layer between the active material electrode layer and the less active layer, the middle layer incorporating at least one nano- and/or micro-scale material; wherein electricity is generated by the sensing component to power the electronics component when moisture comes into contact with the middle layer; and an endpoint node for receiving a signal transmitted by the electronics component when powered by the sensing component.

In another aspect, the endpoint node is a smartphone, tablet or laptop. In a further aspect, the at least one self-powered sensing device includes at least two sensing devices for creating a mesh network. In yet another aspect, the at least one self-powered sensing device is integrated within a piece of clothing, a band-aid, a diaper, a custom-wearable device or a bedsheet.

In yet a further aspect of the disclosure, there is provided a method of manufacturing a self-powered moisture sensing device including creating a sensor component by creating an active material electrode layer; depositing a middle layer atop the active material electrode layer; and placing a passive electrode layer atop the middle layer; and electrically connecting an electronics components to the sensor component; whereby power generated by the sensing component when exposed to moisture is transmitted to the electronics component to power the electronics component.

In another aspect, the depositing a middle layer includes compacting graphite powder into a flat layer of graphite powder, the flat layer of graphite representing a graphite middle layer; and pressing the graphite middle layer atop the active material electrode layer. In an aspect, the creating an active material electrode layer includes polishing a surface of the active material electrode layer before pressing the graphite middle layer onto the active material electrode layer. In yet another aspect, the method further includes hydrophilic treating the middle layer. In a further aspect, the hydrophilic treating the middle layer occurs before depositing the middle layer atop the active material electrode layer. In another aspect, the hydrophilic treating the middle layer occurs after depositing the middle layer atop the active material electrode layer. In yet another aspect, depositing the middle layer atop the active material electrode layer is performed by vacuum filtration or electrophoretic deposition.

In yet a further aspect, the middle layer comprises a matrix or compacted structure of nano- or micro-scale materials that can absorb moisture from an ambient gas and that has at least one nanoscale or microscale dimension. In another aspect, the active material electrode layer includes elemental metals and their alloys which react with non-oxidizing acids at room temperature, but do not combust in a reaction with water or oxygen at room temperature in an air ambient at normal atmospheric pressure.

### DESCRIPTION OF THE DRAWINGS

Embodiments of the present disclosure will now be described, by way of example only, with reference to the attached Figures.

FIG. 1a is a set of schematic diagrams of a self-powered moisture/gas/fluid enabled sensor;

FIG. 1b is a perspective view of a sensing component of the self-powered moisture/gas/fluid enabled sensor;

FIG. 1c is a perspective view of a sensing component as part of an experimental setup;

FIG. 1d is a perspective view of another embodiment of a sensing component;

FIG. 1e are views of a sensing component housing;

FIG. 2a is a flowchart outlining a first method of manufacturing a self-powered moisture/gas/fluid enabled sensor;

FIG. 2b is a flowchart outlining another method of manufacturing a self-powered moisture/gas/fluid enabled sensor

FIG. 3 is a flowchart outlining another method of manufacturing a self-powered moisture/gas/fluid enabled sensor sensor;

FIG. 4a is a flowchart outlining another method of manufacturing a self-powered moisture/gas/fluid enabled sensor sensor;

FIG. 4b is a flowchart outlining a further method of manufacturing a self-powered moisture/gas/fluid enabled sensor sensor;

FIG. 4c is a flowchart outlining another method of manufacturing a self-powered moisture/gas/fluid enabled sensor sensor;

FIG. 5 is a schematic diagram of a moisture sensing experimental setup;

FIG. 6a is a graph showing open-circuit voltage (OCV) pulses generated in response to a humidity change for one embodiment of a self-powered moisture sensor with a middle layer of carbon nano-fibers;

FIG. 6b is a graph showing short-circuit current (SCC) pulses generated in response to a humidity change for the self-powered moisture sensor used in FIG. 6a;

FIG. 7a is a graph showing OCV pulses generated in response to a humidity change for a TiO₂—Mg alloy self-powered moisture sensor;

FIG. 7b is a graph showing SCC pulses generated in response to a humidity change for the self-powered moisture sensor used in FIG. 7a;

FIG. 8a is a graph showing OCV pulses generated in response to a humidity change for another embodiment of a CNP—Mg alloy self-powered moisture sensor;

FIG. 8b is a graph showing SCC pulses generated in response to a humidity change for the self-powered moisture sensor used in FIG. 8a;

FIG. 9a is a graph showing OCV pulses generated in response to a humidity change for another embodiment of a graphene-Mg alloy self-powered moisture sensor;

FIG. 9b is a graph showing SCC pulses generated in response to a humidity change for the self-powered moisture sensor used in FIG. 9a;

FIG. 10a is a graph showing the OCV pulses generated in response to human breath using one embodiment of a self-powered moisture sensor manufactured using the method of FIG. 3;

FIG. 10b is a magnified graph of a single OCV pulse of FIG. 10a;

FIG. 10c is a graph showing SCC pulses generated in response to human breath for the self-powered moisture sensor used in FIG. 10a;

FIG. 10d is a magnified graph of a single SCC pulse of FIG. 10c;

FIG. 11a is a graph showing the OCV pulses generated in response to human breath using another embodiment of a self-powered moisture sensor

FIG. 11b is a magnified graph of a single OCV pulse of FIG. 11a;

FIG. 11c is a graph showing SCC pulses generated in response to human breath for the self-powered moisture sensor used in FIG. 11a;

FIG. 11d is a magnified graph of a single SCC pulse of FIG. 11c;

FIG. 12 is a schematic diagram of a system of water leak detection;

FIG. 13 is a schematic diagram of a system for water leak detection with a mesh network;

FIG. 14a is a schematic diagram of another embodiment of a system for moisture detection;

FIG. 14b is a schematic diagram of a further embodiment of a system for moisture detection;

FIG. 15a is a table showing different test examples of a graphite middle layer;

FIG. 15b is a graph showing power density for the graphite middle layers shown in FIG. 15a;

FIG. 15c is a table showing a comparison of peak power to surface area for graphite middle layer sensors;

FIG. 15d is a chart showing voltage generated vs thickness for the graphite middle layers of FIG. 15a;

FIG. 15e is a graph showing results for water level sensitivity for a graphite middle layer;

FIG. 15f is a graph showing results for temperature sensitivity for a graphite middle layer;

FIG. 15g is a graph showing results for a sensor having stacked graphite middle layers;

FIG. 15h is a table showing test results longevity testing for a sensor with a graphite middle layer;

FIG. 16a is a table showing a first set of test results of how a sensor with a graphite middle layer responds to different types of urine;

FIG. 16b is a table showing a second set of test results of how a sensor with a graphite middle layer responds to different types of urine;

FIG. 16c is a table showing a third set of test results of how a sensor with a graphite middle layer responds to different types of urine;

FIG. 16d is a graph showing results of voltage vs source current for different urine samples;

FIG. 16e is a graph showing results of voltage vs source current for different urine samples at a temperature of 35° C.; and

FIG. 16f are graphs showing results of how a sensor with a graphite middle layer reacts to different concentrations of urine samples.

### DETAILED DESCRIPTION OF THE EMBODIMENTS

The disclosure is directed at a moisture, gas or fluid-enabled sensor or sensing device and method of making same. The sensor of the disclosure may be seen as self-powering, as will be described in more detail below. In one embodiment, the sensing device includes a sensing component and an electronics component whereby when the sensing component is exposed to moisture, humidity, gas or a fluid (seen as moisture”), electricity may be generated by the sensing component to power the electronics component. In the following description, the word “moisture” may refer to liquids, fluids such as oil, blood, urine, water, pure liquid water or an aqueous mixture (e.g. alcohol-water mixture, CO₂-water mixture, human breath mixture, etc.) in the form of vapors (e.g., humidity, fogs, mists, wetness, etc.), carbon dioxide, molecular species in the biophysical environment such as human breath or gases such as, but not limited to, ammonia or carbon monoxide. Furthermore, the term “moisture” may also refer to the physical phases of liquid, gas, and the mixture of liquid and gas, which could be composed of small fluid or water droplets and fluid or water molecules. These small fluid or water droplets and molecules may accumulate on a surface to form a liquid water layer. In the following descriptions, the word “moisture” may be replaced with the word “gas” or the word “fluid”.

In one embodiment, the present disclosure is directed at a self-powered humidity, moisture, gas or fluid enabled sensing device that includes an active metal electrode or active metal electrode layer (e.g. magnesium and/or aluminum or other like materials) that acts as an anode or anode electrode, a porous hydrophilic middle layer (e.g. carbon nanofibers, TiO₂ nanowires, Al₂O₃ nanoparticles, polymers with nano/micro scale channels and/or graphite or other like materials) and a less active electrode or less active electrode layer (e.g. carbon and/or copper or other like materials) that acts as a cathode or cathode electrode. In one embodiment, the anode and cathode electrodes are directly connected by the middle layer (made from materials with nano and/or micro-scale porosity without the addition of separators and/or external electrolytes). When the middle layer is exposed to and/or absorbs moisture/gas/fluid, the nano- or micro-scale materials acts to connect the two electrodes, and reacts with the anode metal to generate voltage/current signals in whose amplitudes are proportional to moisture concentration and humidity levels.

The generation of power is based on an electrophysical and/or electrochemical reaction between the active metal electrode layer and moisture absorbed by the middle layer.

In one embodiment, the middle layer may be made from porous hydrophilic nano- and/or micro-scale materials in direct contact with the active metal electrode, without adding external electrolytes. The middle layer may also be a composite of different materials.

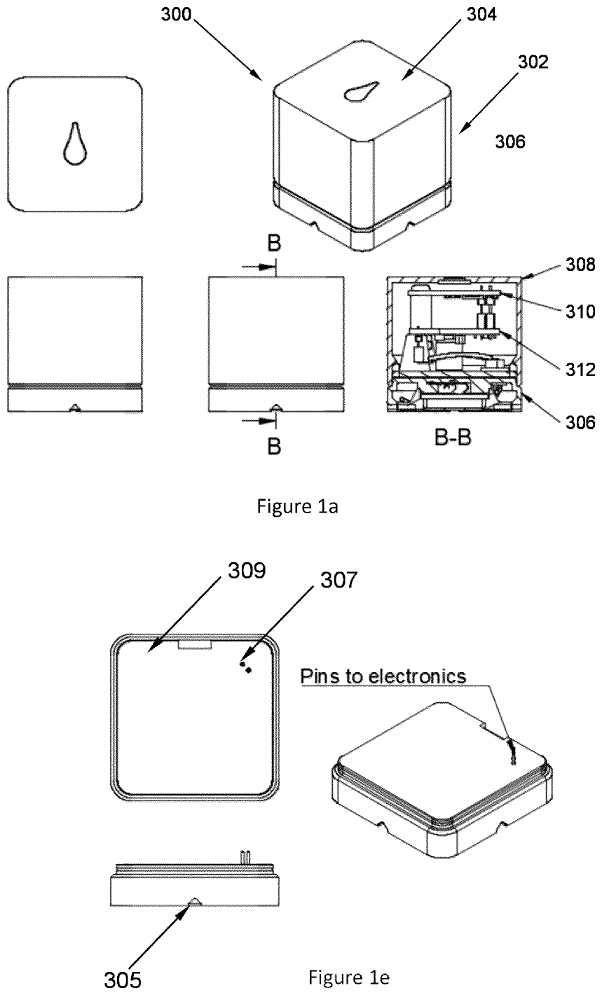

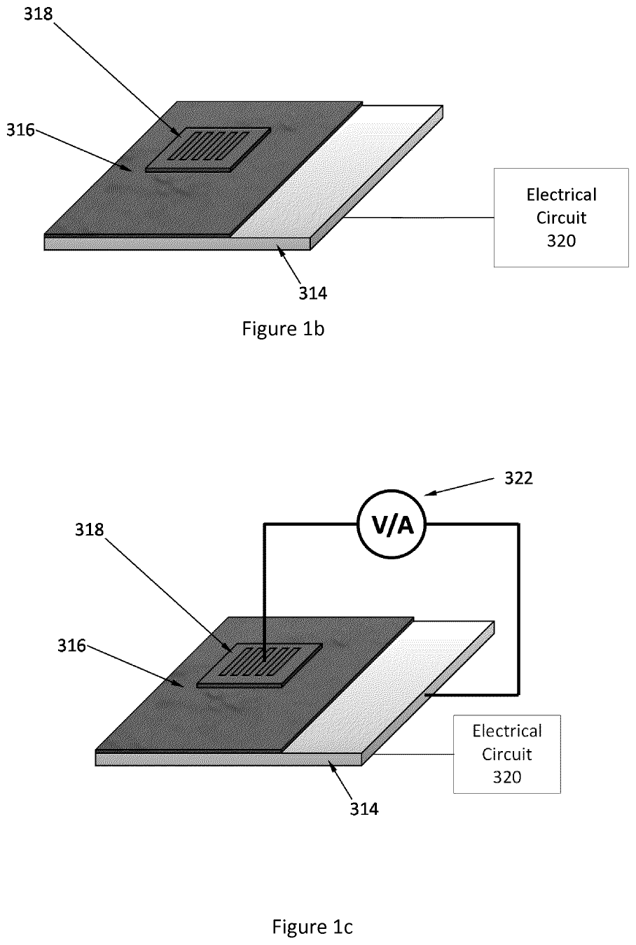

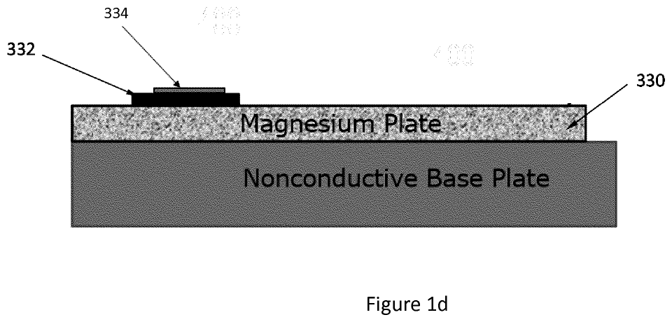

Turning to FIG. 1a, a schematic view of a moisture, gas or fluid-enabled sensor or sensing device is shown. As will be described, the sensor may be seen as self-powering.

The sensor 300 includes a housing 302 that houses an electronics component 304 and a sensing component 306. In one embodiment, the housing 302 may be two separate housings, each housing one of the electronics component and the sensing component. The electronics component 304 may include analysis or application specific components that enable the sensor 300 to process measurements or readings obtained by a sensing component 306 or to analyze the moisture, liquid or fluid that has been sensed. In another embodiment, the electronics components 304 includes communication hardware enabling the sensor 300 to communicate with or transmit signals or information to an external, or remote, device, such as, but not limited to, a user computing device, a cellphone or an endpoint node. The electronics component 304 may also include a combination of the application specific components and communication hardware.

As shown in FIG. 1a, in one embodiment, the electronics component 304 includes an electronics package 308 which may include circuits that interface with the sensing component 306, a low-energy or low-power wireless radio such as a Bluetooth™ low energy (BLE) device 310 and a boost converter 312. Other examples of a low-energy wireless radio may include SigFox™ or LoRa™ radios. One example of an electronics package may be Nordic Semiconductor's nRF52832 chipset. As will be discussed below, the sensor 300 may be seen as self-powering as the sensing component 306 generates power, or electricity, when it comes into contact with, or adsorbs, moisture. This will be described in more detail below.

FIG. 1e provides several views of a housing for the sensor component. In one embodiment, the housing 309 for the sensor component 306 is designed such that there is an electrical contact between the layers of the sensing component (as discussed below). Also, the housing 309 may provide protection to the fragile layers of the sensing component so that the electrical contact between the layers does not fall apart. Also, the housing is designed such that moisture can easily contact the middle layer.

As shown in FIG. 1e, at a top surface of the housing, a set of, in the current embodiment two, pins 307 extend out of the housing 309 for connection, or transmission, of the power generated by the sensing component to the electronic components. In one embodiment, the pins 307 may be connected with the electronics package 308. The housing 309 further includes a set of holes 305, in the current embodiment triangular, to draw moisture from outside the sensing component into the sensing component. In one embodiment, the sensing component may include filter paper inside the housing that assists to direct moisture, such as water or other fluid, towards the components within housing. In one embodiment, the housing for the electronics component and the housing for the sensor component may be held together via an annular snap fit. This enables either component to be easily replaced when needed without needing to replace the entire sensor.

Turning to FIG. 1b, a perspective view of one embodiment of the sensing component is provided. The sensing component 306 includes a set of different layers including an active material, or metal, electrode layer 314, a middle layer 316 and a passive electrode, or less active, electrode, layer 318. In one embodiment, the middle layer can be a composite of different materials. The sensing component 306 may further include an electrical circuit 320 that stores electricity generated by the sensing component 306. In another embodiment, the electrical circuit 320 is connected to the electronics component 304 and provides power to the electronics component 304 to power at least one of the electronics package 308, the Bluetooth™ low energy (BLE) device 310 and/or the boost converter 312 or other parts within the electronics component. In one embodiment, the active material electrode layer 314 and the passive, or less active, electrode layer 318 are in direct electrical contact with the middle layer 316 without the addition of an electrolyte. In the current disclosure, the term less active is being used with respect to the level of chemical and/or physical reaction of the material of the less active electrode layer with respect to the level of chemical and/or physical reaction of the material of the active material electrode layer with the sensed or adsorbed moisture, gas or fluids. The range of power that is generated by the sensor component may be based on different factors, such as, but not limited to, the design of the layers of the sensing component, the power requirements of the electrical components, the moisture being sensed or the application(s) of the sensor.

In one embodiment, the middle layer 316 is made up of nano- and/or micro-scale materials and may be seen as a nano- or micro-scale material layer. The middle layer may include a matrix or compacted structure of nano- or micro-scale materials that can absorb moisture from an ambient gas and that has at least one nanoscale or microscale dimension. The middle layer 316 may also be composed of a single material layer, multiple material layers or a mixture of different materials. The middle layer is located between, and preferably in electrical contact with, the active material electrode layer 314 (seen as the bottom layer) and the less active electrode layer 318 (seen as the top layer). It is understood that top and bottom are being used for explanation purposes and that the location of the active material electrode layer and the less active electrode layer with respect to the middle layer may be reversed in some embodiments.

The middle layer may also be seen as a porous hydrophilic layer whereby porous may be defined as a matrix or a compacted structure of nano- and/or micro-scale material that contains nano- and micro-channels between individual nano/microstructures rendering the middle layer porous to moisture and facilitating the transmission of moisture from the less active electrode layer to the active material electrode layer. Example materials for the middle layer may include, but are not limited to, carbon nano-fibers, graphite, CNP, graphene and TiO₂ nanowire thin layers. While one property of the middle layer is that it is hydrophilic, depending on the material being used, the middle layer may require a treatment of its surface to render it hydrophilic, for instance when the material is CNF, carbon nanostructures and the like. This surface treatment may include, but is not limited to, exposure to an oxygen plasma treatment and/or acid oxidation. The hydrophilic characteristic of the middle layer enables water or moisture to be more easily absorbed on its surface, and can easily wet and spread along the surface of the porous middle layer to connect the two electrode layers.

In one embodiment, the less active electrode layer 318 may be a copper (Cu) mask, however, other materials are contemplated such as conductive materials which are less reactive with water, or moisture, than the material from which the active metal electrode layer is composed. The less active electrode layer may be a single layer, a multilayer or a mixture of these materials. In another embodiment, the shape of the less active electrode layer 318 is designed to expose the nano- or micro-scale material, or middle, layer to moisture. The shape may be a spatially configured mask, as discussed above, or may be a tip electrode whereby a Cu electrode may be terminated in a shaped Cu tip. The active material electrode layer 314 may be composed of a material such as, but not limited to, magnesium (Mg), Aluminium (Al) or Iron (Fe) or alloys of these elements or other materials that may facilitate a reaction between the active material electrode layer and moisture (or fluids, such as water). The active material electrode layer may be a single layer, a multilayer or a mixture of the materials listed above. In another embodiment, the active material electrode layer 314 includes elemental metals and their alloys which react with non-oxidizing acids at room temperature, but do not combust in a reaction with water or oxygen at room temperature in an air ambient at normal atmospheric pressure.

In one embodiment, when in use, the middle layer 316 provides an ionic electrical conduction path between the less active electrode layer 318 and the active material electrode layer 314 when exposed to moisture. The absorption of moisture by the sensing component 306 triggers a reaction between the active material electrode layer and moisture (such as water) that results in the generation of a voltage difference between the two electrode layers thereby producing or generating a current in the electrical circuit 320. The electrical circuit is connected to the electronics component (such as via the pins) whereby the power and characteristics of the generated electrical power, such as output voltage and output current, power the hardware within the electronics component. In one example, the generated power may directly power communication and data storage devices within the electronics component, permitting data transmission to a remote source without an external power source. In another embodiment, the generated power may power other application specific sensors that perform an analysis on the detected moisture.

As shown in FIG. 1c, for testing purposes, the sensing component 306 may further include, or be connected to, a multi-meter 322 for testing output voltage and current signals. The multi-meter 322 is connected between the less active electrode layer 318 and the active material electrode layer 314. This will be discussed in more detail below with respects to the experiments.

Turning to FIG. 1d, a further embodiment of a sensing component for use in a self-powered moisture sensor or sensing device is shown. In the current embodiment, the sensing component 306 includes an active material electrode layer 330, a middle layer 332 and a passive electrode, or less active electrode, layer 334. In the current embodiment, the middle layer 332 is a graphite powder that is press-compacted in a polymer stamp to form porous, water absorbent electrodes that are then layered (as the middle layer) onto the active material electrode layer 330 seen as a magnesium alloy sheet. In other embodiments the graphite powder may be other carbon materials or a powder of other carbon materials. More specifically, the active material electrode layer 330 may be made from a set of magnesium alloy sheets. Water (or moisture) absorbed by the graphite middle layer passes through the porous channels and contact the magnesium layer. In the current embodiment, the middle layer is disc-shaped, although the shape may be changed. The less active layer may be in the form of a clip or mask such as discussed above.

In one embodiment, the graphite middle layer 332 may be fabricated using Aldrich-Sigma 20 um Synthetic powdered Graphite. The graphite powder is formed into a solid disk shape. The middle layer 332 may be any diameter and/or thickness depending on the application of the sensor and/or the power requirements. The active material electrode layer 330 may be manufactured by polishing a set of Mg alloy sheets such that the active material electrode layer 330 has at least one polished surface.

When packaged together as a sensor component, a size of the compartment is selected such that it may reduce or eliminate the swelling of the graphite layer when absorbing the moisture or in other words to continuously compress the graphite layer to improve the functionality of the sensor.

In an experiment, water was delivered onto a top surface of the graphite, or middle, layer, and the voltage output measured and recorded as the sourcing current (the current generated by the sensing component) was varied. The voltage was also recorded when steady state voltage was achieved. The load resistances were varied to achieve different sourcing currents to simulate actual loads from electronic components, such as a voltage boosting circuit or transmitter circuit. By changing the sourcing current, the power output of the graphite-magnesium sensor changed non-linearly as the power output did not scale proportionately to the overall resistance of the circuit. Hence, a range of currents were tested to obtain an overview of how the power varies with current.

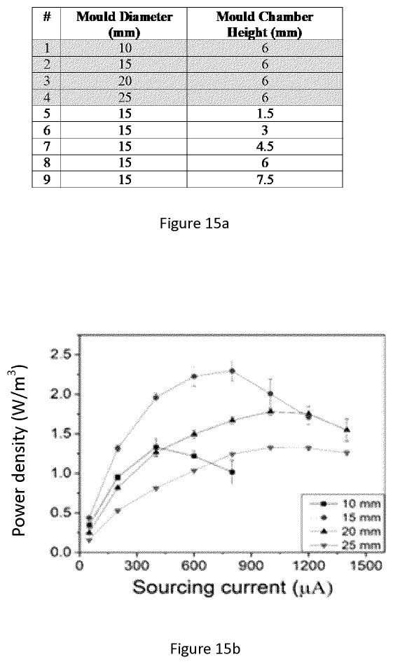

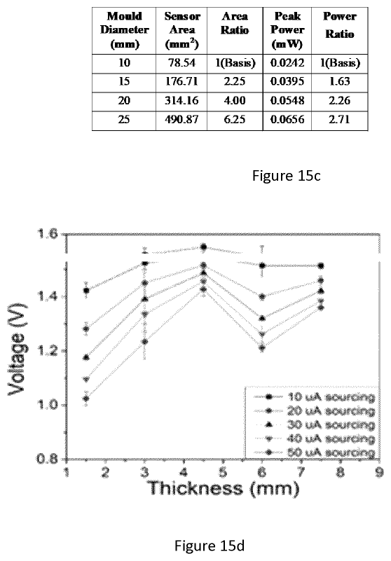

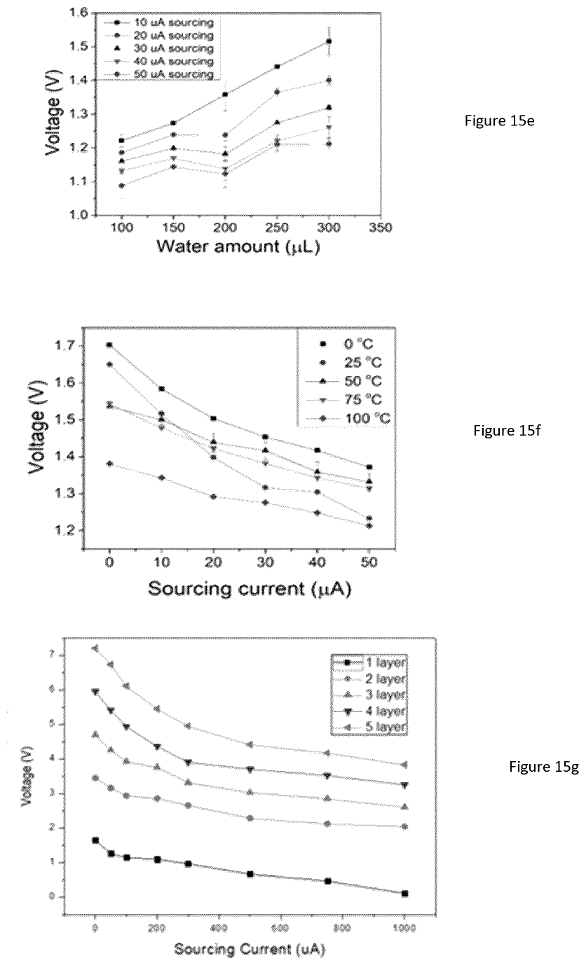

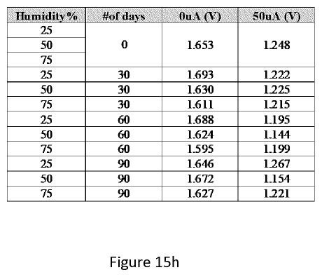

In the current experiment, middle layers with different diameter and thicknesses were tested. A table showing the different graphite middle layer characteristics is shown in FIG. 15a.

FIG. 15b is a graph showing calculated power density with sensors of varying diameter and a thickness of 6 mm (Embodiments 1 to 4 of FIG. 15a). As can be seen, increasing the diameters of the graphite disc or layer increases power output due to the increase in surface area in contact with the magnesium or active material electrode layer. As shown in FIG. 15c, which is a table showing a comparison of peak power to surface area, the power output increase sharply when the diameter of the middle layer is greater than 20 mm. In one embodiment of the disclosure, the diameter of the middle layer is approximately 15 mm to approximately 20 mm.

For a real-world application usage, the load and current draw on the sensing component also factors into the power output as the peak power are at different sourcing current values ranging from ˜400 μA to ˜1100 μA. Selecting a suboptimal power-output diameter for use with a wireless transmitter may be needed to ensure optimal or improved functioning of the sensor. For example, using a 15 mm diameter graphite layer within the sensor may output the best power for its surface area but may fail to function after approximately 1200 μA sourcing current. Some ultra-low resistance electronic components may be able to draw more power from a large-diametered sensor if the combined load results in a cell current of >1200 μA. At 24.2 μW, even the smallest sensor size (or middle layer diameter) tested achieved enough power to charge and maintain a Bluetooth transmitter chip at optimum power. However, this is assuming optimum power draw which will change depending on the components of the electronic components used in the sensor. The internal resistance of the sensor itself, which depends on the amount of moisture in the graphite disc or middle layer, would also affect power output.

FIG. 15d shows the voltage generated vs thickness of the middle layer. With a diameter of 15 mm (embodiments 5 to 9 of FIG. 15a), different sourcing currents (which is the current generated by the sensing component when exposed to water or moisture) were measured ranging from between 10 to 50 μA to obtain thickness measurements. In the experiment, it was determined that a thickness of 4.5 mm provided the best results. It is understood that with other thicknesses, power was generated and therefore, while 4.5 mm was a preferred thickness for the embodiment of the current experiment, other thicknesses may be considered or contemplated that fall within the scope of the disclosure. Therefore, one operational embodiment of the sensor as determined by the experiment is a middle layer having a 4.5 mm thickness with a 15 mm diameter.

For testing of this embodiment, its sensitivity was tested using a water amount ranging from 100 μL to 400 μL with load requirements of 10 μA to 50 μA. Results of this testing is shown in FIG. 15e. The tests show a marked drop in voltage generated when the water amount was reduced with critical values at 250 μL which presumably is approximately the amount required to saturate the 15 mm diameter graphite disk with sufficient water such that approximately all points of contact between the middle layer and the magnesium layer have been exposed water. When 100 μL amount of water was used, this amount of water was still sufficient to power the Bluetooth transmitter as long as sourcing current was above 20 μA. Sensitivity of the device is hence not limited to large volumes of water and can be used in applications which require sensitivity down to the micrometre scale of single droplet size.

With respect to temperature, temperature-dependent behaviour of the magnesium-graphite sensor was tested with the sensor temperature between 0° C. to 100° C. The results are shown in FIG. 15f. As seen in FIG. 15f, while temperature does affect voltage and hence, power output, this relationship is not linear. This may be due to the internal resistance of the sensor component increasing when the temperature increases. This may also be due to the fact that while the intermediate temperatures 25-75° C. show approximately similar outputs, the extremes at 0 and 100° C. show maximum, or high, and minimum, or low, voltages respectively at all sourcing currents. At 100° C., it is possible that the water vaporizes on contact with the graphite or middle layer such that the amount of water added is insufficient and far below the 250 μL threshold hence causing lower power output than expected. At 0° C., resistance of the entire setup is kept at the lowest possible value among the temperatures tested and hence displays the highest voltage values.

Experiments using stacked middle and active layers were also performed. Multiple sensor layers were stacked vertically to achieve a series configuration and tested by inserting identical amounts of water to each layer. Total voltage output was then plotted as shown in FIG. 15g. For the experiment, stacking was performed by cutting square (2 cm×2 cm) blocks of magnesium substrate. These blocks were then layered with conductive copper tape on one side to prevent or reduce the likelihood of two-surface Mg-water reactions which would result in zero net voltage output. The covered blocks were then placed copper-tape side downwards onto the graphite middle layers.

As shown in FIG. 15g, at higher layer counts or layers, there exists some loss in expected voltage likely due to water seepage through the copper top layer. However, stacking in this manner does boost overall output and can potentially be used in sensing devices that do not require μL-sensitivity and can meet higher power requirements. However, if a template structure can be used to hold the layers in place to prevent or reduce compression of the graphite layers as well as electroplating of an inert metal onto one side of the magnesium plates, it is likely that the layers at 3 to 5 will display additive voltages rather than showing diminishing returns as above. Layers 1 to 2 show that the stacking can be achieved with minimal loss in power output as the curve at layer 2 is approximately double that of the single layer structure.

A long-term experiment was also set up and performed over a period of 90 days to check if the sensor with the graphite middle layer could withstand storage under high humidity conditions without a decay in performance. This test was performed by setting graphite layers onto the magnesium alloy sheets and placing them in calibrated humidity chambers under constant controlled humidity. The samples were removed from the humidity chamber in batches of 2 and tested as per the standard experimental procedure. This was performed every 2 weeks for a total of 6 separate instances spread out over 90 days. Different humidity levels were tested as well. FIG. 15h shows the list of tests performed as well as the average steady state result at 0 μA and 50 μA sourcing currents. Temperature and humidity were measured at 25° C. and 25% for all cases. There was no correlation between humidity and time spent in the humidity chamber with the voltage output of the sensor. Degradation of the sensor even in humidity as high as 75% was not detected and sensors having a graphite middle layer placed in those circumstances could still operate within the power requirements of electronic components, such as the Bluetooth board.

When the complete system was tested, the sensor was able to create a wireless signal after 75 seconds. The wireless transmission was detected on a nearby smartphone. As such, it can be seen that the sensor can operate or function both as a power source and a leak detecting sensor, and that the sensor can successfully be integrated into a reliable packaging with the required electronics for operation.

In one embodiment, operation of the self-powering aspect of the sensor 300 or sensing component 306 is based on the redox reaction between the active material electrode layer 314 and the sensed moisture or fluid, and the electrophysical/electrochemical interactions between the middle layer and the moisture/fluid. When the active metal layer (such as active material electrode layer 314) is connected with the less active electrode layer 318 via the moisture/fluid, electrophysical and/or electrochemical reactions occur which generate electricity. This generated electricity is stored in the external or electrical circuit or may be delivered directly to the electronics component. This electricity may then be used by the electronic component 304 to transmit signals, such as via the BLE device 310, to an endpoint node, such as a user device (tablet, laptop, Smartphone™) or to analyze the detected moisture or other applications.

In use, the active material electrode layer 314 oxidizes (when contacted by moisture or water) yielding positive ions that migrate towards the cathode via a current in the fluid, while the free electrons travel from the Anode electrode to the cathode electrode via the external circuit where H+ ions in the H₂O combine with electrons to produce hydrogen gas. The OH— ions in H₂O combine with metal positive ions at the cathode to form hydroxide. Typically, these reactions are so rapid that the hydroxide and hydrogen gas produced may cover the electrode surface and hinder further reaction. Therefore, it is necessary to change the nucleation and deposition positions of these reactions enabling the sensor of the disclosure to operate more smoothly.

When moisture, such as water, is sensed by the sensor component 306 whereby it has entered the sensor 300, there are three regions in the metal-fluid-metal structure that can affect ionic conductivity. These may be seen as (a) the Anode-fluid interface; (b) the interior of the fluid and (c) the cathode-fluid interface.

Processes in these three regions greatly influence ion transportation within the sensing component 306. By inserting the nano- or micro-scale material, or middle layer 316 between the two electrodes (the less active electrode layer 318 and the active material electrode layer 314), different functions or functionality can be achieved. One of these functions is the absorption of the detected moisture for use as a fluid to connect the active electrode and the less active electrode, and form an inner circuit that generates the electricity. Another function is the formation of conducting paths for water (or moisture) on the hydrophilic surface of the nano-material layer 316 to accelerate the transportation of ions. The ionic conductivity of nano- or micro-scale materials determines the internal resistance and output power.

A third functionality is that the sensing component may serve as nucleation and deposition sites for hydroxide materials and for hydrogen gas, allowing side products to be absorbed resulting in the continuous exposure of a fresh Anode surface. In addition, the contact resistance between the nano- or micro-scale material of the middle layer 316 and the electrodes 318 and 314 determines whether or not an internal short circuit is produced at the two interfaces between the nano-material layer 316 and the less active electrode 318 and the nano-material layer 316 and the active material electrode layer 314. If the electronic conductivity is comparable to the ionic conductivity, some electrons will travel along the nano- or micro-scale material, reducing the output power.

In some embodiments, in order to make a middle layer with hydrophilic nano/micre-scale porous structure, some hydrophilic treatments may need to be performed to some materials that are not hydrophilic in nature. In this embodiment, the nano/micro-scale porous structures are made by nano/micre-scale materials. Nano/micro-scale materials like graphite or TiO₂ nanowires are usually hydrophilic in nature, while materials like carbon nanofiber or graphene are hydrophobic.

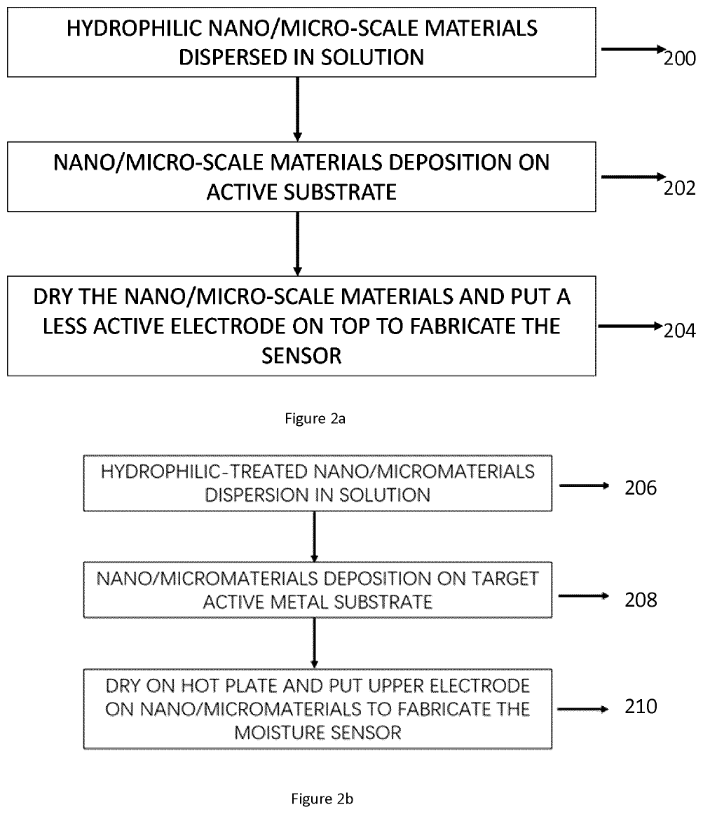

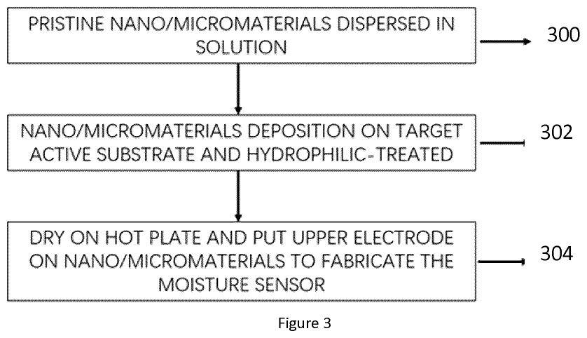

Turning to FIGS. 2a, 2b and 3, flowcharts showing different methods of manufacturing the self-powered moisture sensor are shown. In some embodiments, the method may be selected based on the material of the middle layer.

Turning to FIG. 2a, the method may be seen as one that is for manufacturing a self-powered sensor with a middle layer made from a hydrophilic material. Initially, hydrophilic nano/micro-scale materials are dispersed in a solution (200). The nano/micro-scale materials are then deposited on an active substrate, or an active material electrode layer (202). The nano/micro-scale materials may be seen as the middle layer of the sensor. The nano/micro-scale materials are then dried and a less active electrode layer is placed on top of the nano/micro-scale materials, or middle layer.

In the embodiment of FIG. 2b, the method may be seen as one that is for manufacturing a self-powered sensor that is hydrophilic-treated before deposition. In this method, the material used for the middle layer is hydrophobic. In the embodiment of FIG. 3, the method may be seen as one that is for manufacturing a self-powered moisture sensor that is hydrophilic-treated after deposition. As such, the difference between these two processes is the sequence of deposition and hydrophilic treatment.

Initially, as shown in FIG. 2b, hydrophilic-treated nano or micro scale materials are dispersed in a solution (206). The nano or micro materials (seen from this point forward as nanomaterials) are then deposited (as a nanomaterial layer or the middle layer) on an active metal substrate or layer, or the active material electrode layer (208). The deposition may be performed via electrophoretic deposition, vacuum filtration or moulding to control the thickness and porous morphology of the nanomaterial layer although other deposition methodologies may be contemplated. The term “porous morphology” refers to a structure which contains nanoscale or microscale channels between single nano- or micro-scale material units. The methods of FIGS. 2a, 2b and 3 may be adopted to form a porous nanoscale material middle layer. The combination is then dried, such as on a hot plate, and a top, or passive electrode layer integrated with the nanomaterial layer (210).

As shown in FIG. 3, the nanomaterials are initially dispersed in a solution (300). Example solutions may include any aqueous solution or ethanol solution where the solvent can be either water or an organic solvent. The nanomaterials are then deposited on the active material electrode layer and hydrophilic-treated (302). Possible deposition methods are discussed above with respect to FIG. 2b. The combination is then dried, such as on a hot plate, and an upper, or top, electrode integrated with the nanomaterial layer (304).

Special treatments to produce an enhanced hydrophilic surface improve the adsorption of moisture. These include, but are not limited to, plasma treatment and acid oxidation. By adding oxygen functional groups on the surface of the nano- or micro-scale materials, hydrogen bonds can be formed more easily between nano- or micro-scale materials and water molecules. For example, pristine CNF is hydrophobic, but become hydrophilic after oxygen plasma treatment. Some nano- or micro-scale materials are intrinsically hydrophilic like TiO₂ nanowires, which are materials for sensing moisture.

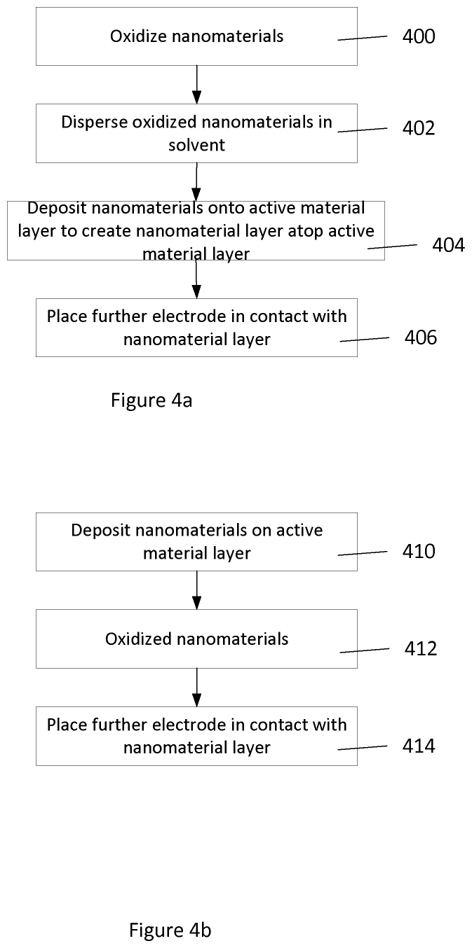

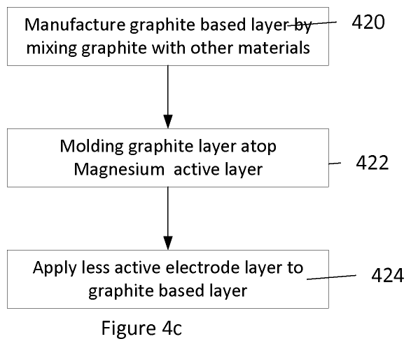

Turning to FIG. 4a, a flowchart outlining another method of manufacturing a self-powering moisture sensor is shown. Initially, nano- or micro-scale materials are first oxidized with an oxidizing agent (400) such as nitric acid (HNO₃) or potassium permanganate (kMnO₄). The pre-oxidized nano- or micro-scale materials are then dispersed in a solvent (402), such as by ultrasonic vibration, in order to separate the nano- or micro-scale materials into small pieces to increase the porosity and surface to volume ratio after deposition. In one embodiment, the solvent may be water, alcohol, isopropanol, or acetone. A thin film or layer of nano- or micro-scale materials is then deposited, or formed on an active material layer (404) such as by electrophoretic deposition or vacuum-filtration.

In one embodiment of electrophoretic deposition, the active metal layer, or active electrode material layer, and a counter passive electrode are inserted into the solvent, and the distance is tuned to achieve an optimal, or predetermined, electric field intensity between the electrode and the counter electrode. A voltage bias of 10-30V is then applied between these two electrodes, and the charged nano- or micro-scale materials suspended in the colloidal solution, or solvent, migrates toward the substrate. Applying this bias for 1 min forms a homogeneous network of nano- or micro-scale materials containing an abundance of interstitial nano/microchannels. The thickness of the nano- or micro-scale materials network can be readily controlled by the applied voltage or by varying the deposition time. For example, a solution with 0.1 wt % CNF is used for CNF deposition on Mg alloy, and a 0.1 mm thick film is achieved after deposition for 1 min with a 30 V bias voltage.

In the process of vacuum deposition, the prepared solution is vacuum-filtered into nano/micro-networks of different thickness by tuning the solution concentration, vacuum pressure, as well as the size of single nano- or micro-scale material units (particles, wires, flakes). In one exemplary embodiment, 10 mg CNF, with an average diameter of 130 nm and 20-200 nm in length, is vacuum filtered into a slice that is 15 mm in diameter and 0.3 mm thick. Following deposition, the substrate was coated with a uniform layer of nano- or micro-scale material and then annealed at 100° C. for 12 h to improve the adhesion between the nanomaterial network and the substrate.

The nano- or micro-scale material layer is then placed in contact with another electrode, or the less active electrode layer (406), such as Cu to complete fabrication of the moisture sensor.

Turning to FIG. 4b, another embodiment of a method of manufacturing a moisture sensor is shown. Initially, the pristine nano- or micro-scale materials (or middle layer) are deposited on a target electrode (410), such as the active material electrode layer. The nanomaterial layer is then oxidized (412), such as by oxygen plasma. The nano- or micro-scale material layer is then in placed in contact with another (the less active) electrode layer or material (414), such as Cu.

For the sensors manufactured in the flowcharts of FIGS. 2, 3, 4a and 4b, the nano- or micro-scale material moisture sensors are based on the combination of an active material electrode layer made from or composed of Mg alloys, a middle layer of nano- and/or micro-scale materials (including, but not limited to, carbon nanofibers (CNF), carbon nanoparticles (CNP), graphene flakes, or TiO₂ nanowires) and a Cu passive or less active electrode/wire. While these materials form a specific embodiment for experimental testing, it is to be understood that other materials may be utilized, and structural changes may be made without departing from the scope of the disclosure.

Turning to FIG. 4c, a further embodiment of a method of manufacturing a self-powering sensor is shown. Initially, a layer of graphite is created or manufactured (420). In one embodiment, the graphite layer may be disc-shaped and created by placing graphite powder in a press mould and then compressing the powder together. The graphite layer may also be manufactured by mixing graphite with other materials. The graphite layer is then layered atop an active electrode material layer of magnesium alloys (422). In one embodiment, Mg alloy sheets may be polished, and the graphite disc is then lightly pressed onto the polished surface of the active material electrode layer. A less active layer is then put onto the graphite layer (424).

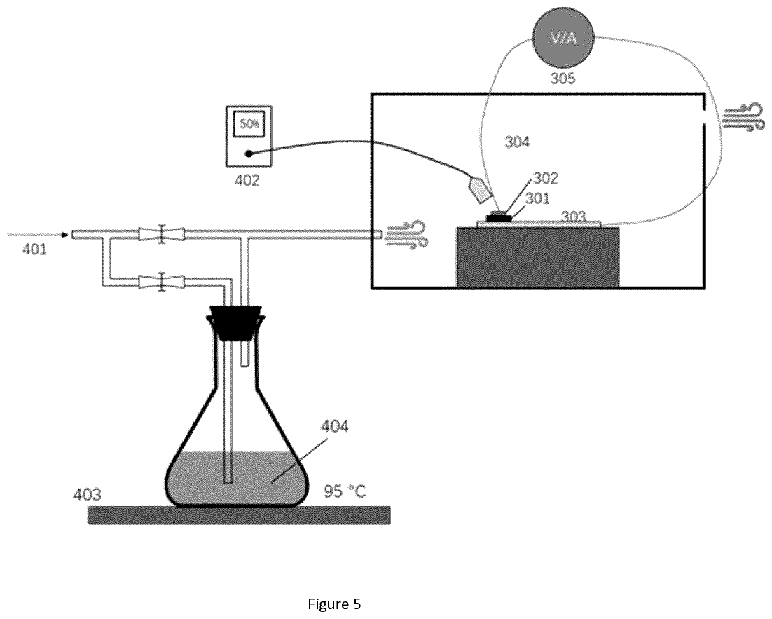

FIG. 5 provides a schematic diagram of one embodiment of a setup for an experiment for sensor embodiments relating to the flowcharts of FIGS. 2a, 2b, 3, 4a and 4b. The sensor 300 was placed within a humidity controlled chamber 500 that included an inlet 500a and an outlet 500b. In the experiment, a multi-meter 501 is connected between the top electrode layer 318 and the active material electrode layer 314. A humidity sensor 502 was also placed within the chamber 500. The setup for the experiment further included a beaker or container 504 containing water 506 that was placed atop a hot plate 508. For the experiment, the hot plate was set at 95° C. A set of tubes 510 connected the container 506 with the inlet 500a of the chamber 500. Compressed air 512 was also introduced into the tubes 510.

In the experiment, the open circuit voltage (OCV) and SCC signals of a Mg-0.1 mm PTCNF-Cu moisture sensor in response to humidity changes were tested in the humidity-controlled chamber. Wet air and dry air was blown into the sealed chamber successively to make the humidity within the chamber 500 increase and decrease.

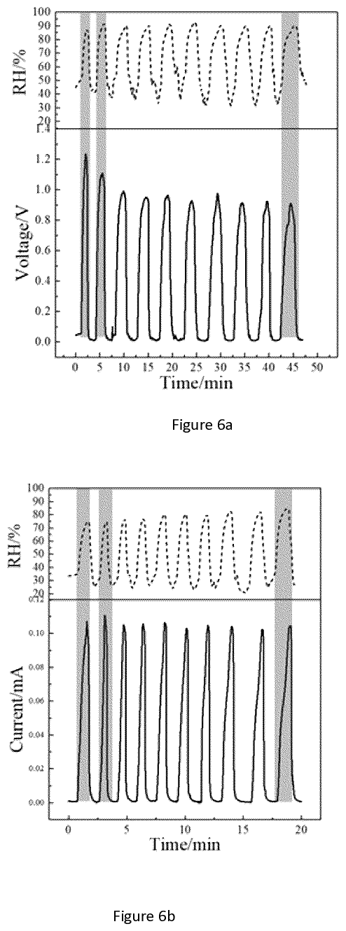

FIGS. 6a and 6b show how humidity influences the OCV and SCC, respectively. For the current experiment, a PTCNF-Mg based device was used. The super-hydrophilic and porous surface of the CNF efficiently absorbs H₂O molecules from the air, transferring them to the PTCNF-Mg interface where they react. As the CNF layer is thin enough, moisture can diffuse more rapidly to the Mg surface. This PTCNF-Mg based device was sensitive to environmental humidity changes and responded well to changes in moisture concentration. An OCV of ˜1V and a SCC of ˜100 μA were reproducibly achieved over a period of 20 min.

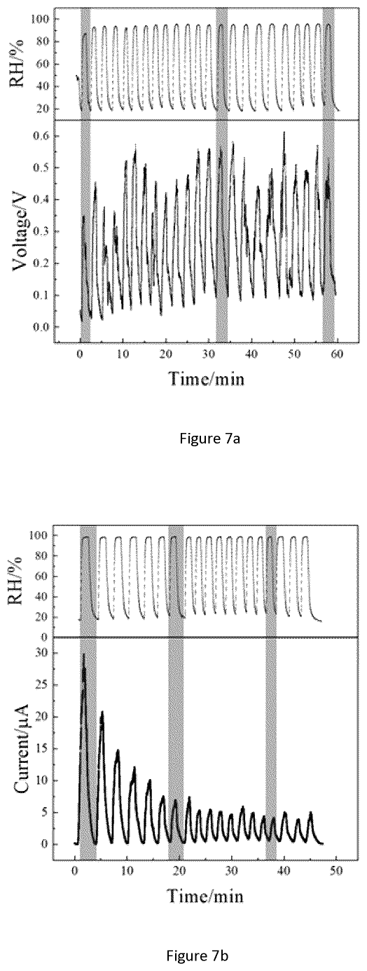

In addition to CNF, other nanomaterials were used as the nanomaterial middle layer. TiO₂ nanowire is an insulating nanomaterial, and is intrinsically hydrophilic. Carbon nanoparticles (CNP) and graphene are hydrophobic, and need plasma treatment after deposition to make them hydrophilic. FIGS. 7, 8 and 9 show the OCV and SCC for TiO₂—Mg alloy, CNP—Mg alloy and graphene-Mg alloy devices, respectively, in response to humidity changes.

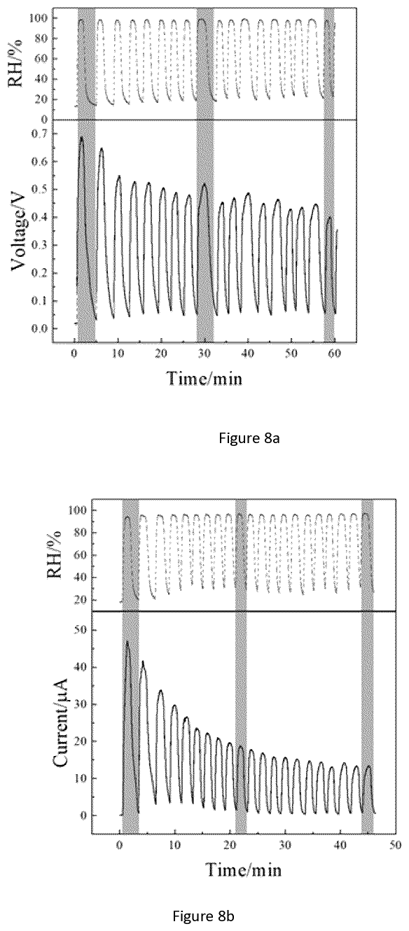

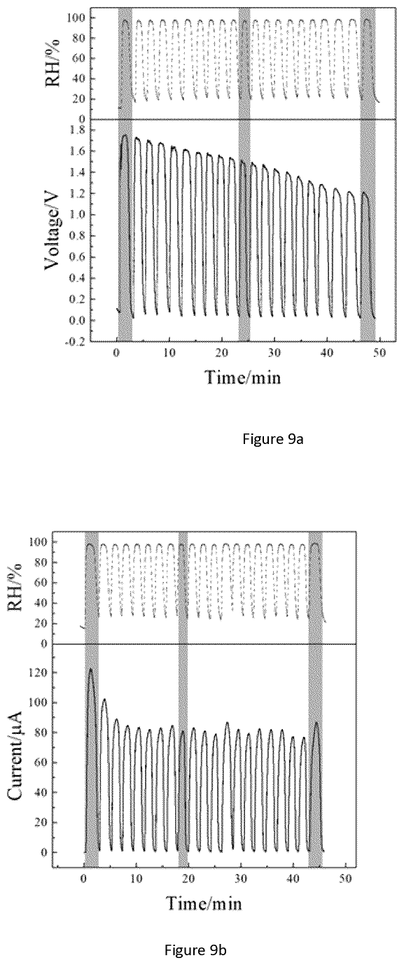

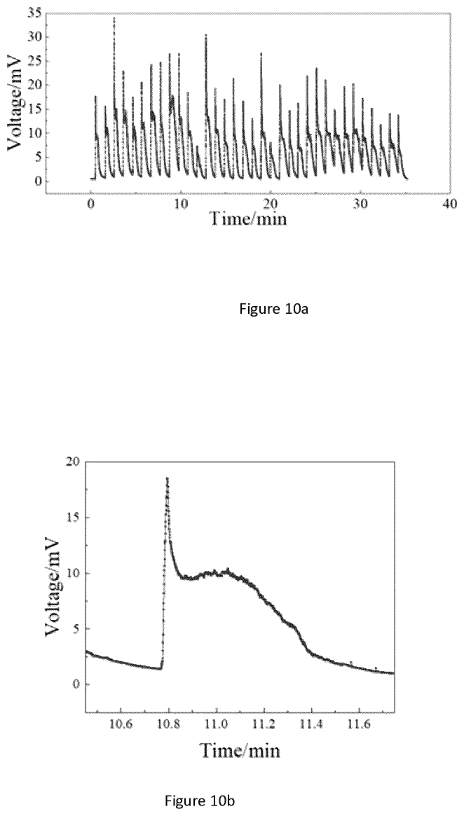

FIG. 7a shows that an OCV of ˜0.5V can be achieved, but that this value was not stable. FIG. 7b shows that the SCC decreased from 30 μA to about 5 μA, and then remained constant. This means that the performance of TiO₂ devices may degrade with time, however, they are still able to generate electricity to power the electronics component. Similar results were obtained with CNP devices. FIG. 8a shows the OCV increased to 0.7V at first and then slowly decreased to about 0.4V, while the SCC reached 47 μA and quickly decreased to 15 μA (FIG. 8b). For graphene-Mg alloy devices, the OCV was as high as 1.7V, and then slowly decreased to 1.3V (FIG. 9a), while the initial current was 120 μA, and then fell to a constant value of about 80 μA (FIG. 9b). In summary, the devices based on zero-dimensional (CNP), one-dimensional (CNF, TiO₂ nanowires) and two-dimensional (graphene) nanomaterials can all generate voltage and current in response to changes in moisture concentration and humidity. Not only insulating nanomaterials (TiO₂ nanowires) but also conductive materials (CNF, CNP, graphene) can serve as the nanomaterial middle layer 316. According to the above experiment results, the OCV and SCC signals of the CNF—Mg device, and the SCC signal of graphene remain stable at high values for a long time and are in the correct range for use for sensing moisture and humidity levels.

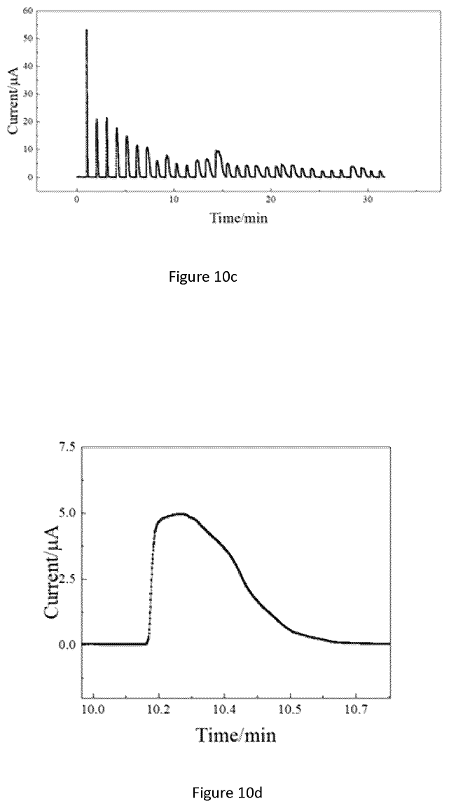

A further experiment was performed with Mg—CNF—Cu and Al—CNF—Cu moisture sensors made by the process of FIG. 3. In this experiment, the sensors were used for breath sensing. The CNF films (or nanomaterial layer) were made by vacuum-filtration with a thickness of about 0.3 mm. FIGS. 10a to 10d show the open-circuit voltage (OCV) and SCC (SCC) pulses that were generated by the sensors when exposed to human breath using the 0.3 mm thick vacuum-filtered PTCNF samples with a Mg alloy. To standardize these measurements, the breath signal was collected every minute so that the device, or sensor, was able to dry out between pulses.

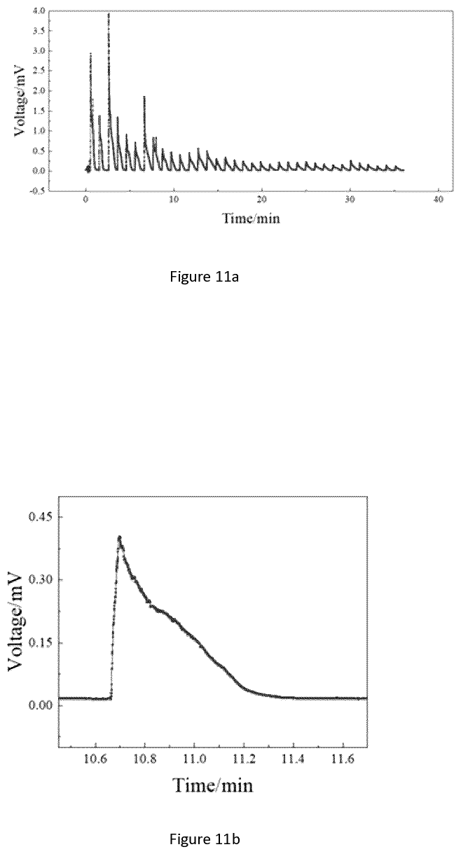

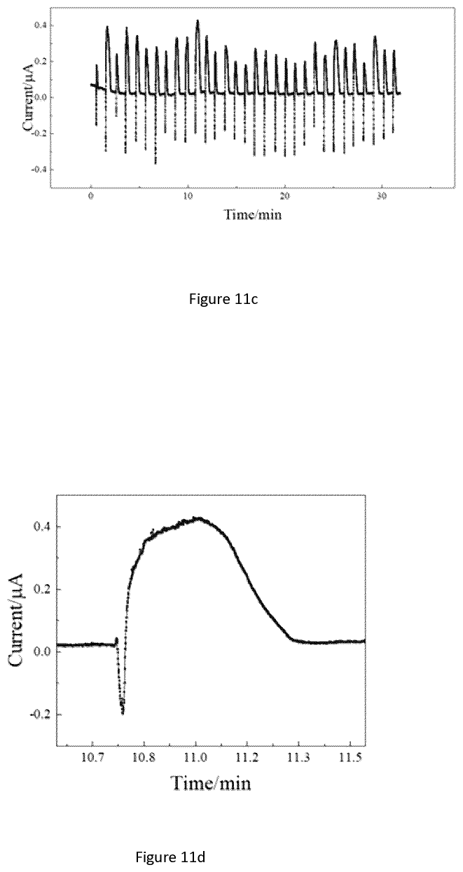

It can be seen that the PTCNF-Mg device generated a voltage of around 20 mV and a current of around 50 μA in response to each breath. The peak voltage output over an extended time was stable whereby the peak pulse voltage remained constant over a 30 min period and showed good repeatability (FIG. 10a). The current pulses were high at first but then quickly decreased to a steady value of 5 μA (FIG. 10c). FIG. 10b shows that, for each pulse, the V-t pattern shows a fast discharge peak followed by a longer signal due to the water reaction. The signals can be separated into prompt and delayed components. The prompt components are caused by the reduction of oxygen groups on the CNF surface, while the delayed components are caused by the water reaction. The time dependence of the capacitive discharge component of the OCV curve is described by U=0.020 exp(−t/1.84). For comparison, the voltage and current pulses from plasma-treated CNF—Al samples are given in FIG. 11, and it can be seen that the voltage and current generated by Al is about one order of magnitude smaller that from Mg, and the voltage discharge peak became less significant (FIGS. 11a and 11b). It was also observed (as shown in FIGS. 10c and 10d) that the current discharge peak is negative. The negative and positive signal peaks in these sensors are assumed to correspond to streaming potentials generated by moisture diffusing in and out of the device. The highly sensitive, reproducible response of these devices in breathing tests indicates that they are well suited to potential applications as breath sensors.

The electrochemical reactions can be triggered and controlled by the moisture absorbed by porous, hydrophilic nano- or micro-scale material middle layer, and voltage and current signals generated in response to changes of moisture concentration and humidity are sensitive to the presence of the gases detected. From experimentation, it was determined that highest open-circuit voltage was about 1.7V, and the highest SCC was about 120 μA. These outputs are sufficient to power many low-powered remote communication and data storage devices. In addition, this device also showed a high sensitivity to human breath, and generated different signal amplitudes when constructed with Mg and Al substrates.

Different applications of the sensor are contemplated and discussed below. It is understood that other applications are contemplated whereby there is a need for a moisture sensor detector. The self-powering feature of the moisture sensor detector of the disclosure provides an advantage over current sensors.

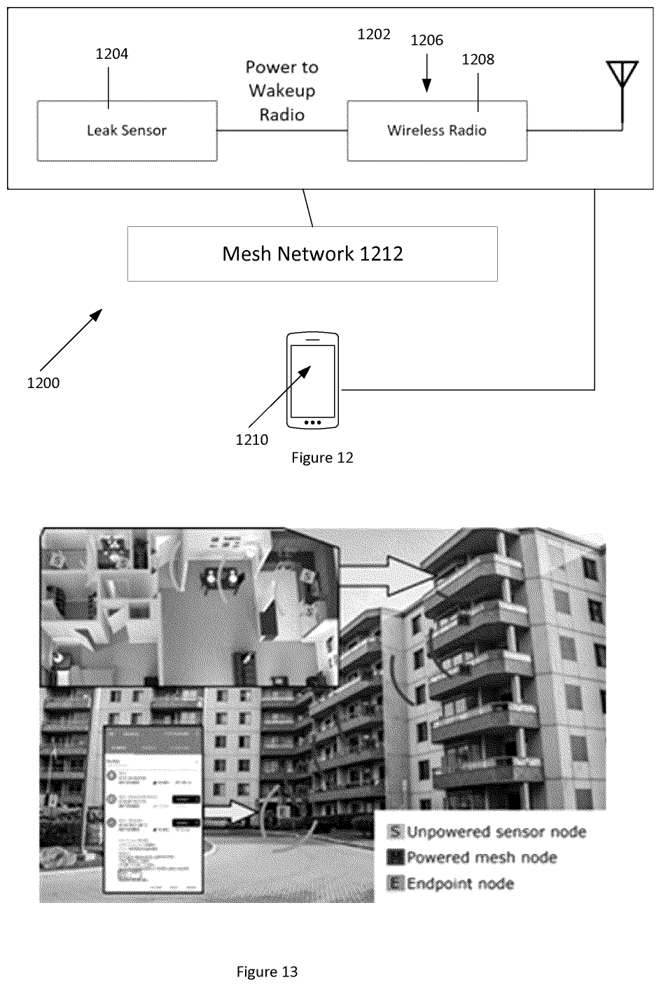

In one application, or embodiment, the self-powered moisture/gas/fluid enabled sensor may be used as part of a water leak detection system. Turning to FIG. 12, a schematic diagram of a water leak detection system is shown. In one embodiment, this may be used to detect water leaks in a home or other building. While FIGS. 12 and 13 are directed at a water leak detection system, the detection system may also be used to detect moisture, humidity and/or gas.

The system 1200 includes a sensor 1202 that includes a sensor component 1204 and an electronics component 1206 such as the one described above. The electronics component 1206 may include a radio component 1208 that can communicate, wirelessly, with a user device 1210. The radio component 1208 may be a standard wireless radio with power interface and general purpose input/output interface lines along with analog to digital (A/D) converters; a wireless capable chip that has a power interface connected to a sensor output whereby the radio component wakes up and transmits data only when the sensors detects a leak that enables it to generate enough power to activate the radio component; a wireless radio that uses RFID or Bluetooth connectivity; a wireless radio that uses custom wireless connectivity; a radio that uses an integrated antenna, a radio that includes a flexible antenna; or a radio that uses the sensor as an antenna.

Depending on the application, the sensor may be a water sensor; a liquid sensor; a fluid sensor; a moisture sensor; a humidity sensor; a carbon monoxide sensor; a carbon dioxide sensor; an oil sensor; a gas sensor; or a multi-functional sensor combining any of the aforementioned sensors.

In operation, when water is detected by the sensor component 1204, electricity is generated by the sensor that causes the radio component 1208 of the sensor 1202 to “wake up”. The sensor 1202 may then transmit a warning message or signal to the user device 1210 indicating that it has detected the presence of water in its vicinity. While only one sensor 1202 is shown in FIG. 12, it is understood that a plurality of sensors may be provided that communicate with the same user device 1210 where a mesh network 1212 of sensors may be created. In one embodiment, the sensor 1202 may communicate with other sensors in the mesh network to transmit the signal to the user device 1210.

In another embodiment, the system of FIG. 12 may be seen as the application of a BLE mesh networking with a water detection sensor that includes custom energy harvesting circuitry to power the sensor when water comes into contact with the sensor. Each sensor then becomes a notification device. In this embodiment which may be referred to as a Beacon-Mesh Integration (BMI) system, the sensors send out a Bluetooth Low Energy (BLE) beacon while they are powered (by the presence of water). This beacon is identified by proximal powered mesh network nodes, which then create and send mesh messages. When the messages reach a preselected endpoint mesh node (or nodes), various reactions are possible (such as a WiFi-enabled board sending a message to a server). In the current embodiment, the endpoint node may generate a beacon of its own which can be identified using the smartphone.

In another embodiment, multiple sensor and wireless radio combinations are embedded together to form the mesh network. In a further embodiment, the sensor and the radio component are integrated on a compact printed circuit board.

In some embodiments, the sensor 1202 may include a transmitter wireless radio connected to the sensing component 1204; a transmitter or radio component that is powered by the sensor only in the presence of water; a radio component that operates for a very limited amount of time; whereby the radiation from the transmitter or radio component is very limited in terms of power or duration, posing no health risk; a receiver radio component that receives the alert signal coming from sensor; a receiver radio component for alerting the building owners/operators/maintenance worker via a mobile application, an automated phone call, or a text message; or a receiver radio component that may relay the alert to another radio or server to increase the range of coverage and insure leak detection and alert over larger distances

The system may also include different self-powered sensor systems, which are connected together to sense different variables (i.e. water and gas).

In another example, it may be desirable to have a self-powering leak detection system that detects water leak in buildings which in turn help to reduce water damage and insurance claim. The current system may provide the further advantage of a leak detection system that is capable of leak detection and notification without relying on repetitive wireless transmission, thus reducing the cost and simplified installation. In terms of implementing a water leak detection system with Beacon-Mesh Integration (BIM) feature in an apartment building, in one embodiment, each apartment may include one powered, fully featured mesh node. Therefore, instead of requiring each sensor in a unit to be powered, only a single powered node is required to support many sensors. Assuming that the sensors in the apartments are within a predetermined range of one another, a single large mesh network covering an entire apartment building would be created. An endpoint device could be placed anywhere in the building (such as in a maintenance office) as long as it is in range of at least one other mesh device.

Another advantage of the current system is the uniqueness of overall architecture of the system is unique which includes a power generating sensor that is self-powered and a simplified low-cost wireless radio.

In another application, or embodiment, the self-powered moisture/humidity/liquid sensor may be used as part of a battery-free wearable wireless sensor system. More specifically, the sensor may be used to detect urine in an individual's clothing or bed sheets. In one embodiment, the sensor may be part of a system that detects wet diapers or underwear for infants and/or older adults to help avoid many of the related health complications. One advantage over some current systems is that the system may function without the need for batteries. Another advantage of using the self-powered sensor described above in such a system is that it does not rely on repetitive wireless transmission, thus potentially reducing exposure to harmful wireless radiation.

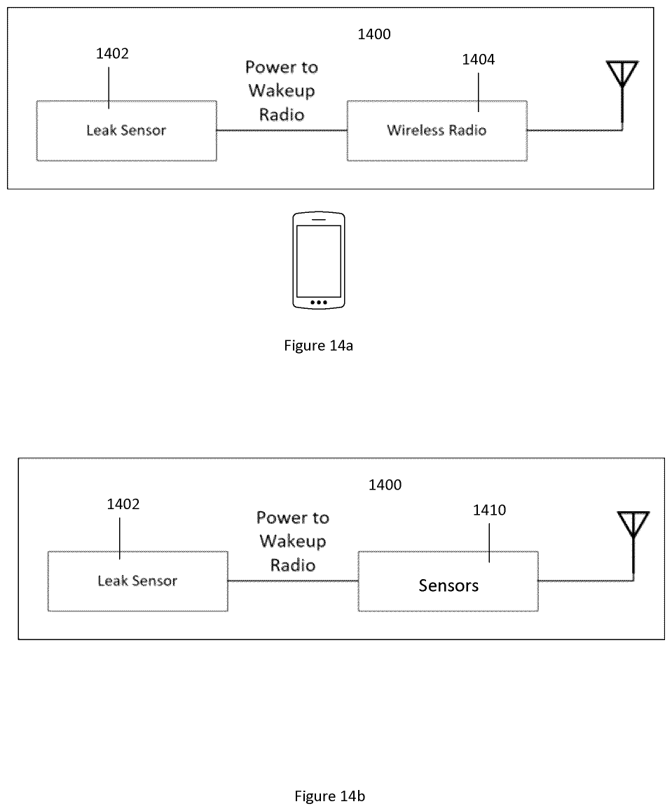

Turning to FIG. 14a, a schematic diagram of a battery-free system for detecting wet clothing is shown. The system 1400 includes a sensor component 1402 that is connected to, integrated with, or associated with a, preferably wireless, radio component 1404. The radio component may have a transmitter component and a receiver component. In one embodiment, conductive ink may be used to interface the sensor component 1402 with the radio component 1404. In one embodiment, the sensor component 1402 may be integrated in the diaper material or clothing (T-shirt, jeans, pants) of an individual, a band-aid or bed sheets. In an alternative embodiment, as shown in FIG. 14b, the wireless radio component may be replaced by sensors 1410, or application specific sensors, that perform analysis of the sensed moisture such that the power generated by the sensor component may be used for powering components that enable testing of the sensed moisture. For instance, the sensors 1410 may include blood testing apparatus or urine testing apparatus. In other embodiments, the application specific sensors may include, but are not limited to, a humidity sensor, a lactate sensor, a mineral sensor, a temperature sensor, a glucose level sensor, a urine analysis component or a blood analysis component. In another embodiment, the sensing component may power a urine analysis component or sensor within the electronics component to determine if the user has renal dysfunction by testing a presence of phosphates.

As described above, the sensor component includes the components to generate power when it comes in contact or detects a wet diaper. When the sensor component comes into contact with the water/urine, it generates electricity which may then power the radio component. When the radio component is powered, the receiver component of the radio component may sense alert signals that are generated by the sensor component and then transmit a signal to an endpoint, or end node, via its transmitter component. The endpoint may be another radio or server to increase a range of coverage or a smartphone associated with a caregiver (or family member) via a mobile application, an automated phone call and/or a text message.

In the case of a smartphone, the system for detecting a diaper leak may also include an application that is stored on the smartphone to receive signals or alerts from the sensor. The application on the smartphone may communicate with a gateway and a Cloud database to receive the alerts.

In operation, the sensor may sense the presence of, or adsorb, urine which causes power to be generated by the sensing device. This power may then be used to power a radio transmitter to transmit a signal indicating a wet diaper. In one embodiment, a gateway may scan for different transmitted and then and filters the signals to determine which were transmitted by a diaper or moisture sensor or represent a signal indicating a wet diaper. Once an alert signal from a diaper or moisture sensor is detected, the gateway inputs this alert into a database, such as the Cloud database, with the sensor ID and start date information. The Cloud database is used to store a history of diaper leaks.

Concurrently, the application stored on the Smartphone polls the Cloud database on a continuous or pre-determined time interval to determine if there are any entries in the Cloud database that match a sensor ID associated with that Smartphone. If there is a match, the application will alert the user through a push notification whenever a new leak is detected.

In one embodiment, a first tab on the Smartphone application may display the leak status of the diaper (wet or dry) as well as the time when a leak was detected. If the user wants to change the status of the diaper, they may click on the alert symbol and change the leak status to “dry”, in the event that they have changed the diaper. In one embodiment, this will create a new entry with the time the diaper was changed. It is understood that while the current example reflects a one-to-one relationship between the Smartphone and a diaper sensor, it is understood that a single Smartphone may also be associated with multiple diaper sensors.

This implementation currently monitors the leak of one sensor placed in a diaper. The software can be scaled in the future to accommodate for more than a single sensor to be useful in applications such as hospitals or nursing homes where many patients need to be monitored.

In another embodiment of the system for detecting wet clothing, the sensor component and the radio component may be embedded together to form a smart textile diaper/underwear/pants/shirts/etc. . . . . In a further embodiment, the sensor component and the radio component may be integrated on a PCB, whereby the PCB may be rigid or flexible. The PCB materials may also be made of textile materials.

In this application, one advantage of the system of the disclosure is that it provides battery-free wet clothing sensing using a wireless radio that is only powered when detection occurs, thus enjoying very little amount of wireless radiation.

Experiments using the sensor device of FIG. 1d in the detection of a fluid, urine were performed. In the experiment, five different samples of artificial urine were used and seen as Urine Control, Urine Albumin, Urine Phosphate, Urine Glucose and Urine Vitamin C. The Urine Control sample reflects the composition of urine in healthy individuals who have no pathology while the other urine samples reflect the composition of urine mixed with the identified material.

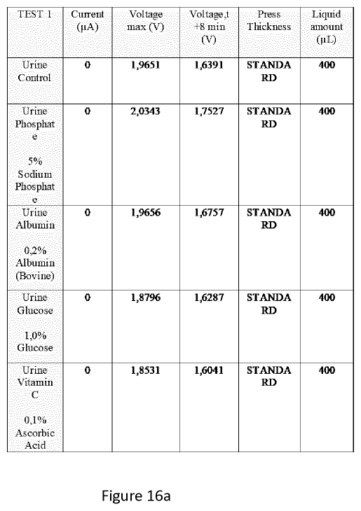

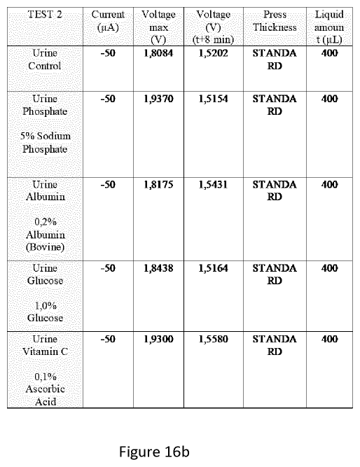

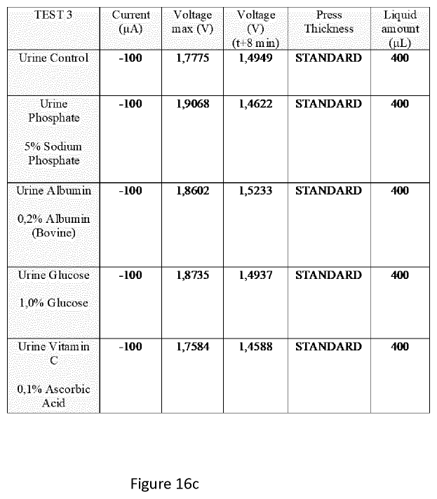

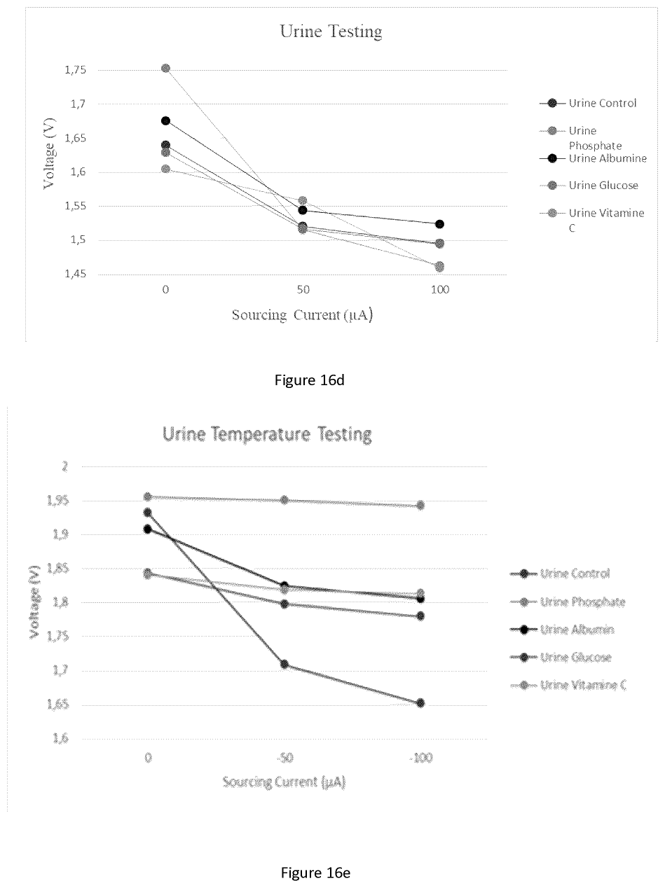

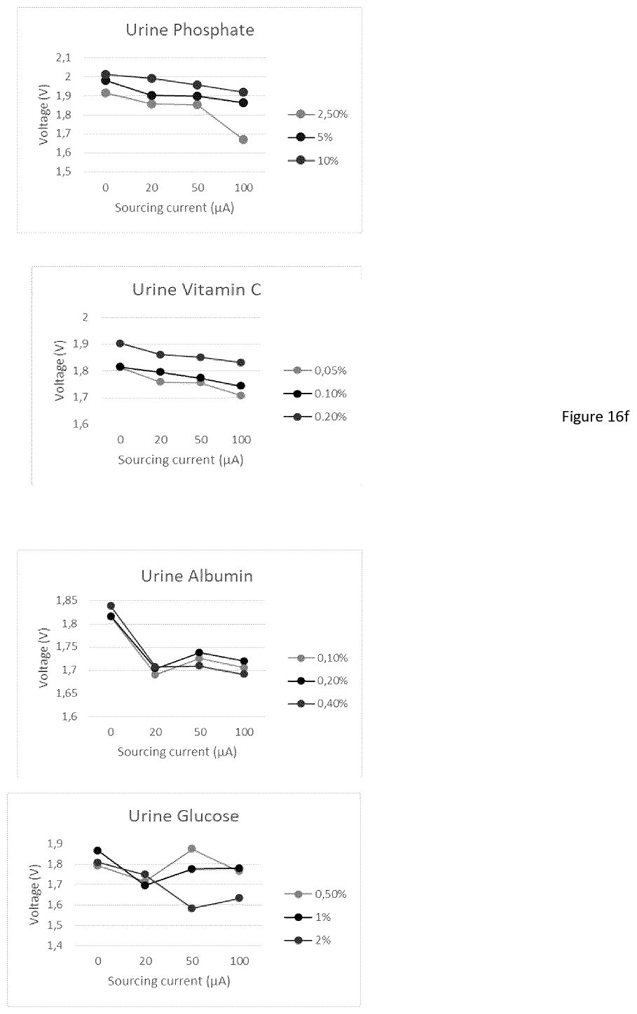

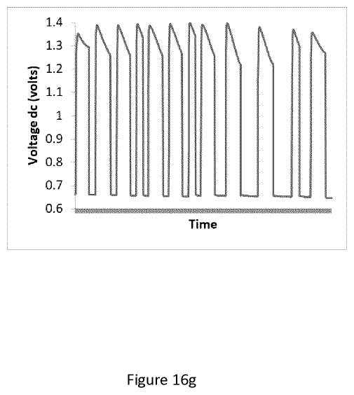

Voltage was measured for each sample using different sourcing currents (0 μA, −510 μA and −100 μA where negative represents power flowing from the sensor device into the source meter). The amount of urine used was 400 μL at 25° C. and humidity at 25%. Results are shown in FIGS. 16a, 16b and 16c. A comparative table of the results is shown below:

| Urine Control | | Urine Phosphate | | Urine Albumin | |
|---|---|---|---|---|---|
| Current | Voltage | Current | Voltage | Current | Voltage |
| (μA) | (V) | (μA) | (V) | (μA) | (V) |
| 0 | 1.639093 | 0 | 1.75268 | 0 | 1.6757 |
| 050 | 1.520227 | 50 | 1.515367 | 50 | 1.543128 |
| 100 | 1.49494 | 100 | 1.46221 | 100 | 1.5233 |

| | |
|---|---|
| Urine Glucose | Urine Vitamine C |

| Current | Voltage | Current | Voltage |
|---|---|---|---|
| (μA) | (V) | (μA) | (V) |
| 0 | 1.6287 | 0 | 1.6041 |
| 50 | 1.516354 | 50 | 1.558 |
| 100 | 1.49368 | 100 | 1.45882 |

As shown in FIG. 16d, for the five case studies, the decrease in voltage is proportional to the decrease in sourcing current: the more the electric current increases the more the voltage decreases. At 0 A, the highest voltage is observed with the Urine Phosphate sample where the voltage approaches 1.8 volts which is considerably higher than when the sensor is activated with water, where it often reaches 1.6 volts. It is likely that the phosphate ions produce some additional reaction with the ions already present in the sensor. It was determined that the sensor was able to generate power based on the artificial urine samples in each of the different types.

In temperature tests (reflecting the true temperature of urine exiting a user's body, a temperature of 35° C. was selected. Again, experiments were run with the 400 μL urine samples at 35° C. with a humidity at 25% at different sourcing currents (0 μA, −510 μA and −100 μA). A comparative table us shown below. FIG. 16e shows the table in graph form.

| | | |
|---|---|---|
| Urine Control | Urine Phosphate | Urine Albumin |

| Current | Voltage | Current | Voltage | Current | Voltage |
|---|---|---|---|---|---|
| (μA) | (V) | (μA) | (V) | (μA) | (V) |
| 0 | 1.9311 | 0 | 1.955 | 0 | 1.907 |
| −50 | 1.7078 | −50 | 1.9494 | −50 | 1.8236 |
| −100 | 1.6508 | −100 | 1.9415 | −100 | 1.8051 |

| | |
|---|---|
| Urine Glucose | Urine Vitamine C |

| Current | Voltage | Current | Voltage |
|---|---|---|---|
| (μA) | (V) | (μA) | (V) |
| 0 | 1.842 | 0 | 1.84 |
| −50 | 1.7969 | −50 | 1.8179 |
| −100 | 1.7788 | −100 | 1.8122 |

In this experiment, the urine phosphate sample generated the highest voltage, however, the sensor device was able to generate a voltage in each of the tests.

As shown in the graphs of FIG. 16f, diluted urine samples were also tested to determine if the sensor would be able to generate power in the presence of these samples. As can be seen, the sensing device was able to generate power in each scenario.

Further experiments were performed with the sensor installed or integrated within a diaper. In one embodiment, the graphite layer and the magnesium layer were installed with a rigid plastic cover and placed in an inner first layer of the diaper. A flexible printed circuit board was also installed in order to capture the power generated by the graphite and magnesium layers. The decision of placement location of the sensor within the diaper may depend on flow dynamics of the diaper, body position within the diaper and where the urine source is placed within the diaper. For experiment purpose, a first flow of 75 ml of urine was added to the diaper and then a second flow of 75 ml of urine was added after 20 seconds. A signal was detected at about 2 minutes of about 1.2V. There was a slight increase in the sensed voltage after about 20 seconds due to the second flow. This is shown in FIG. 16g. Testing showed that in the embodiment where the electronics components included a transmitter, a signal was sent by the senor and received by a smartphone when a voltage of approximately 380 mV was generated by the sensor. The smartphone may include software to display an alert to a user of the smartphone when the sensor senses a presence of urine, or a liquid. In one embodiment, the software may display a green light when the diaper is in a dry state and a red light when the diaper is in a wet state.

In other applications, the self-powered sensor may be used to sense oil leaks in automobiles whereby the electronics components may be integrated with a car's computer system to send alerts when leaks (liquid, fluid or gas) are detected.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether elements of the embodiments described herein are implemented as a software routine, hardware circuit, firmware, or a combination thereof.

What is claimed is:

1. A self-powered sensing device comprising:
an electronics component;
a sensing component, the sensing component including:
an active material electrode layer;
a less active electrode layer;
a middle layer between the active material electrode layer and the less active layer, the middle layer incorporating at least one material with nano- and/or micro-scale structures; wherein electricity is generated by the sensing component to power the electronics component when moisture comes into contact with the middle layer.

2. The self-powered sensing device of claim 1 wherein the middle layer comprises pressed graphite-based powder or graphite.

3. The self-powered sensing device of claim 2 wherein the pressed graphite powder is pressed into a disc-shaped middle layer.

4. The self-powered sensing device of claim 1 wherein the middle layer is porous and hydrophilic.

5. The self-powered sensing device of claim 1 wherein the active material electrode layer and the less active electrode layer are in direct electrical contact with the middle layer.

6. The self-powered sensing device of claim 1 wherein absorption of moisture, gas or fluid by the middle layer generates a voltage difference between the active material electrode layer and the less active electrode layer.

7. The self-powered sensing device of claim 1 wherein the middle layer comprises carbon nanofibers (CNF), carbon nanoparticles (CNP), graphene flakes, graphite or TiO₂ nanowires.

8. The self-powered sensing device of claim 7 wherein the middle layer is treated via a hydrophilic treatment.

9. The self-powered sensing device of claim 8 wherein the hydrophilic treatment comprises an oxygen plasma treatment or acid oxidation.

10. The self-powered sensing device of claim 1 wherein a material of the less active electrode layer is less chemically or physically reactive with respect to moisture compared to a material of the active material electrode layer.

11. The self-powered sensing device of claim 1 wherein the active material electrode layer, the less active electrode layer and the middle layer comprise a single layer of a material or a multi-layer of the material.

12. The self-powered sensing device of claim 1 wherein the active material electrode layer, the less active electrode layer and the middle layer comprise a single or multi-layer of a mixture of materials.

13. The self-powered sensing device of claim 1 wherein the electronics component comprises at least one of a low-energy wireless device, a low-energy wireless communication device, a Bluetooth™ low energy (BLE) device and an application specific sensor.

14. The self-powering sensing device of claim 13 wherein the application specific sensor comprises a humidity sensor, a lactate sensor, a mineral sensor, a temperature sensor, a glucose level sensor, a urine analysis component or a blood analysis component.

15. The self-powered sensing device of claim 13 wherein the low-energy wireless device is powered by absorption of moisture by the middle layer generating a voltage difference between the active material electrode layer and the less active electrode layer.

16. The self-powered sensing device of claim 1 wherein the electronics component comprises:
a radio component.

17. The self-powering sensing device of claim 1 wherein the active material electrode layer comprise magnesium (Mg), Aluminium (Al), Iron (Fe), alloys of Mg, Al or Fe or other materials that facilitate a reaction between the active material electrode layer and moisture.

18. The self-powering sensing device of claim 1 wherein the passive electrode layer comprises copper or conductive materials which are less reactive with moisture than the active material electrode layer.

19. A system for moisture detection comprising:
at least one self-powered sensing devices, the at least one self-powered sensing devices including:
an electronics component; and
a sensing component, the sensing component including:
an active material electrode layer;
a less active electrode layer;
a middle layer between the active material electrode layer and the less active layer, the middle layer incorporating at least one nano- and/or micro-scale material; wherein electricity is generated by the sensing component to power the electronics component when moisture comes into contact with the middle layer; and
an endpoint node for receiving a signal transmitted by the electronics component when powered by the sensing component.

20. The system of claim 19 wherein the endpoint node is a smartphone, tablet or laptop.

21. The system of claim 19 wherein the at least one self-powered sensing device comprises at least two sensing devices for creating a mesh network.

22. The system of claim 19 wherein the at least one self-powered sensing device is integrated within a piece of clothing, a band-aid, a diaper, a custom-wearable device or a bedsheet.

23. A method of manufacturing a self-powered moisture sensing device comprising:
creating a sensor component by:
creating an active material electrode layer;
depositing a middle layer atop the active material electrode layer; and
placing a passive electrode layer atop the middle layer; and
electrically connecting an electronics components to the sensor component;
whereby power generated by the sensing component when exposed to moisture is transmitted to the electronics component to power the electronics component.

24. The method of claim 23 wherein the depositing a middle layer comprises:
compacting graphite powder into a flat layer of graphite powder, the flat layer of graphite representing a graphite middle layer; and
pressing the graphite middle layer atop the active material electrode layer.

25. The method of claim 23 wherein the creating an active material electrode layer comprises:
polishing a surface of the active material electrode layer before pressing the graphite middle layer onto the active material electrode layer.

26. The method of claim 23 further comprising
hydrophilic treating the middle layer.

27. The method of claim 26 wherein the hydrophilic treating the middle layer occurs before depositing the middle layer atop the active material electrode layer.

28. The method of claim 26 wherein the hydrophilic treating the middle layer occurs after depositing the middle layer atop the active material electrode layer.

29. The method of claim 23 wherein depositing the middle layer atop the active material electrode layer is performed by vacuum filtration or electrophoretic deposition.

30. The self-powered sensing device of claim 1 wherein the middle layer comprises a matrix or compacted structure of nano- or micro-scale materials that can absorb moisture from an ambient gas and that has at least one nanoscale or microscale dimension.

31. The self-powered sensing device of claim 1 wherein the active material electrode layer comprises elemental metals and their alloys which react with non-oxidizing acids at room temperature, but do not combust in a reaction with water or oxygen at room temperature in an air ambient at normal atmospheric pressure.
